(19) United States
(12) Patent Application Publication
(10) Pub. No.: US 2025/0345581 A1
(43) Pub. Date: Nov. 13, 2025

(54) Instrument Delivery Device for Extended Instrument Advancement into an Indwelling Catheter

(71) Applicant: Becton, Dickinson and Company, Franklin Lakes, NJ (US)

(72) Inventors: Curtis H. Blanchard, Herriman, UT (US); Evan Barbour VandenBrink, San Mateo, CA (US); John M. Lackey, West Valley City, UT (US); Justin G. Hortin, Farmington, UT (US); Megan S. Scherich, Salt Lake City, UT (US)

(21) Appl. No.: 19/199,448

(22) Filed: May 6, 2025

Related U.S. Application Data
(60) Provisional application No. 63/643,494, filed on May 7, 2024.

(51) Int. Cl. A61M 39/02

(52) U.S. Cl. CPC A61M 39/0247; A61M 2039/0258; A61M 2039/0273; A61M 2039/0279

(57) ABSTRACT

An instrument delivery device for advancing an instrument into a vascular access device indwelling catheter includes an introducer housing comprising a housing coupler at a distal end thereof and an actuator movably coupled to the introducer housing to move the instrument between a first position where the instrument is disposed within the housing and a second position where a distal end portion of the instrument is disposed distally beyond the introducer housing. A lock of the delivery device secures the introducer housing to the vascular access device, with the lock including a lock coupler that mates with the housing coupler, with the lock coupler positioned about the housing coupler such that the housing coupler is positioned within the lock coupler. The lock also includes a blunt cannula and a pair of arms configured to engage the vascular access device and a fluid seal positioned within a lumen of the lock.

### CROSS-REFERENCE TO RELATED APPLICATION

The present application claims priority to U.S. Provisional Patent Application No. 63/643,494 entitled “Instrument Delivery Device for Extended Instrument Advancement into an Indwelling Catheter” filed May 7, 2024, the entire disclosure of which is hereby incorporated by reference.

### BACKGROUND OF THE INVENTION

### Field of the Invention

The present invention is directed to an instrument delivery device for introducing an instrument such as a catheter, guidewire, or probe into an indwelling catheter of a vascular access device.

### Background of the Invention

Vascular access devices (VADs) are used in the medical field to access peripheral vasculature of a patient for purposes of infusion therapy and/or blood withdrawal. Common types of VA Ds include over-the-needle peripheral intravenous catheters (PIVCs), peripherally inserted central catheters (PICCs), central venous catheters (CVCs), and midline catheters. The VAD may be indwelling for short term (days), moderate term (weeks), or long term (months to years).

Instrument delivery devices are often used with VA Ds to deliver an instrument into the indwelling intravenous (IV) catheter thereof, with the instrument delivery device advancing the instrument beyond the tip of the indwelling catheter. The main method by which these devices work is by employing an introducer for inserting a catheter, probe, tube, or other instrument through the catheter lumen of the indwelling catheter, with the introducer being attached to a catheter adapter of the indwelling catheter that provide for insertion of the catheter. For example, the catheter adapter may include a needleless access connector thereon by which the instrument may be introduced to provide access to the indwelling catheter and into the patient's vasculature. In some embodiments, bodily parameters (e.g., blood properties, temperature, oxygenation, etc.) may be sensed upon introducing of the instrument (i.e., a probe). In other embodiments, a fluid transfer may be performed upon introducing of the instrument (i.e., a catheter), such as via a syringe and/or vacutainer being used to inject fluid into the patient or collect blood samples without needing to subject the patient to additional needle sticks.

The introducer of an the instrument delivery device typically includes a housing, an instrument movable within the housing so as to be extendable out therefrom for advancement into the indwelling catheter, and an advancement member or actuator that may be actuated by an operator relative to the housing. That is, the actuator may be moved distally by the operator to cause a corresponding movement of the instrument relative to the housing, such that the instrument may be advanced out from the distal end of the housing and into the indwelling catheter. However, in certain instances, it is recognized that the instrument delivery device may not be able to extend the instrument a desired distance into/through the indwelling catheter. That is, some catheters (e.g., CVCs and MIG catheters) may extend further/deeper into the vascular, and it may be desired to extend the instrument 30-35 mm of reach beyond the tip of the indwelling catheter (e.g., to prevent thrombosis), with it thus being recognized that the current structure of instrument delivery device may not always be able to accommodate this desire. While lengthening the housing of the introducer would enable a corresponding increased length of the instrument within the housing and for a lengthened extension into the indwelling catheter, lengthening the housing can impact usability of the introducer by making it more difficult to handle.

Accordingly, a need exists for an instrument delivery device and introducer thereof that provides for advancement of an instrument into an indwelling catheter, with the introducer configured to extend the instrument out from the introducer by an increased length. The advancement of the instrument out from the introducer by a longer distance is achieved without increasing a length of the introducer, so as to keep the introducer at an acceptable size that allows for easy use thereof.

### SUMMARY OF THE INVENTION

Provided herein is an instrument delivery device for advancing an instrument into an indwelling catheter of a vascular access device. The instrument delivery device includes an introducer having an introducer housing comprising a proximal end portion and a distal end portion, the distal end portion comprising a housing coupler, and the introducer housing defining an inner volume configured to movably receive the catheter. The instrument delivery device also includes an actuator movably coupled to the introducer housing and configured to move relative to the introducer housing to move the instrument between a first position, in which the instrument is disposed within the introducer housing, and a second position, in which a distal end portion of the instrument is disposed beyond the distal end portion of the introducer housing, such that at least a first portion of the instrument may be disposed within the indwelling catheter. The instrument delivery device further includes a lock configured to secure the introducer housing to the vascular access device. The lock includes a lock coupler configured to mate with the housing coupler of the introducer housing to secure the lock to the introducer housing, with the lock coupler configured to be positioned at least partially about the housing coupler, such that the housing coupler is positioned within the lock coupler. The lock also includes a blunt cannula extending out distally from the lock coupler and configured to engage an access connector of the vascular access device, a pair of arms configured to engage the access connector, and a fluid seal positioned within a lumen of the lock that extends through the lock coupler and the blunt cannula.

In some embodiments, the fluid seal is positioned within the lumen adjacent a distal end of the lock coupler, and distal to the housing coupler when mated with the lock coupler.

In some embodiments, the fluid seal is positioned within a portion of the lumen extending through the blunt cannula.

In some embodiments, the fluid seal is positioned at a proximal end of the lumen extending through the blunt cannula.

In some embodiments, the lock coupler comprises a U-shaped snap-fit that engages the housing coupler on opposing sides thereof.

In some embodiments, the actuator comprises a first portion and a second portion, the second portion positioned within the inner volume of the introducer housing and the first portion extending out through a slot formed in the introducer housing and onto a top surface of the introducer housing.

In some embodiments, the actuator is advanceable to the distal end of the introducer housing so that part of the second portion is positioned in the housing coupler, to place the instrument in the second position.

In some embodiments, the first portion of the actuator comprises an engagement member, and wherein the top surface of the introducer housing includes a plurality of ribs thereon that selectively engage an underside of the engagement member to provide for advancement of the actuator along the introducer housing.

In some embodiments, the plurality of ribs extend along the top surface of the introducer housing, to the distal end thereof.

Another instrument delivery device for advancing an instrument into an indwelling catheter of a vascular access device is provided that includes an introducer comprising an introducer housing having a proximal end portion and a distal end portion, with the distal end portion comprising a housing coupler and with the introducer housing defining an inner volume configured to movably receive the instrument. The instrument delivery device also includes an actuator movably coupled to the introducer housing and configured to move relative to the introducer housing to move the instrument between a first position, in which the instrument is disposed within the introducer housing, and a second position, in which a distal end portion of the instrument is disposed beyond the distal end portion of the introducer housing, such that at least a portion of the instrument may be disposed within the indwelling catheter. The actuator further comprises a first portion including an engagement portion movable along a top surface of the introducer housing and a second portion positioned at least partially within the inner volume and joined to the first portion through a slot formed in the top surface of the introducer housing, wherein the engagement member of the first portion is a cantilevered engagement member that extends distally out past the second portion.

In some embodiments, the second portion comprises a cylindrical extension that protrudes out proximally from the first portion.

In some embodiments, with the instrument in the first position, the actuator is positioned such that the cylindrical extension extends out through an opening in the proximal end portion of the introducer housing.

In some embodiments, the instrument delivery device further includes an extension tube coupled to a proximal end of the cylindrical extension, the extension tube moving along with the actuator as the actuator is moved relative to the introducer housing.

In some embodiments, with the instrument in the second position, a portion of the cantilevered engagement member extends over the housing coupler.

In some embodiments, the top surface of the introducer includes a plurality of ribs thereon that selectively engage a tab on an underside of the engagement member to provide for advancement of the actuator along the introducer, and wherein the plurality of ribs extend along the top surface of the introducer, to the distal end thereof.

In some embodiments, the instrument delivery device further includes a lock configured to couple the introducer to the vascular access device, the lock comprising: a lock coupler configured to mate with the housing coupler of the introducer to secure the lock to the introducer; a blunt cannula extending out distally from the lock coupler and configured to engage an access connector of the vascular access device; and a pair of arms positioned on opposing sides of the lock coupler and the blunt cannula and configured to engage the access connector.

In some embodiments, with the instrument in the second position, the cantilevered engagement member of the first portion extends over a portion of the pair of arms of the lock.

Another instrument delivery device for advancing an instrument into an indwelling catheter of a vascular access device is provided that includes an introducer having an introducer housing comprising a proximal end portion and a distal end portion, with the distal end portion comprising a housing coupler the introducer housing defining an inner volume configured to movably receive the instrument. The instrument delivery device also includes a lock configured to secure the introducer to the vascular access device, with the lock comprising a lock coupler configured to mate with the housing coupler of the introducer housing to secure the lock to the introducer housing, a blunt cannula extending out distally from the lock coupler and configured to engage an access connector of the vascular access device, and a pair of arms positioned on opposing sides of the lock coupler and the blunt cannula, each of the pair of arms comprising a first end configured to engage the access connector and a second end actuatable by a user to cause the first end to selectively engage and disengage the access connector. The blunt cannula comprises an extended cannula configured such that 30% or more of the blunt cannula extends out distally past the first end of the pair of arms for engagement with the access connector. The instrument delivery device further includes an actuator movably coupled to the introducer housing and configured to move relative to the introducer housing to move the instrument between a first position, in which the instrument is disposed within the introducer housing and the lock, and a second position, in which a distal end portion of the instrument is disposed beyond the distal end portion of the introducer housing, such that at least a portion of the instrument may be disposed within the indwelling catheter.

In some embodiments, the blunt cannula has a sidewall defining a lumen therethrough, and wherein a thickness of the sidewall is 0.5 mm or more.

In some embodiments, with the instrument in the first position, a distal end of the instrument is positioned within a lumen of the blunt cannula.

### BRIEF DESCRIPTION OF THE DRAWINGS

FIG. 1 is a perspective view of an instrument delivery device, according to an aspect of the disclosure;

FIG. 2 is a top view of the instrument delivery device illustrated in FIG. 1;

FIG. 3 is an exploded view of the instrument delivery device of FIG. 1;

FIG. 4 is a perspective view of a first member of an introducer housing included in the instrument delivery device of FIG. 1;

FIG. 5 is a perspective view of a second member of the introducer housing included in the instrument delivery device of FIG. 1;

FIG. 6 is a rear perspective view of the introducer housing formed by coupling the first member illustrated in FIG. 4 to the second member illustrated in FIG. 5;

FIG. 7 is a front perspective view of the introducer housing illustrated in FIG. 6;

FIG. 8 is a cross-sectional view of the introducer taken along the line 8-8 in FIG. 6;

FIG. 9 is a rear perspective view of a lock included in the instrument delivery device of FIG. 1;

FIG. 10 is an exploded perspective view a catheter, an extension tube, and an actuator included in the instrument delivery device of FIG. 1;

FIG. 11 is a perspective view of the actuator illustrated in FIG. 10;

FIG. 12 is a side view of the actuator illustrated in FIG. 10;

FIG. 13 is a side view of the blood draw device of FIG. 1 in a first configuration, showing positioning of the actuator and catheter support;

FIG. 14 is a side view of the blood draw device of FIG. 1 in a second configuration, showing positioning of the actuator and catheter support;

FIG. 15 is a top view of an instrument delivery device, according to another aspect of the disclosure;

FIG. 16 is an exploded view of the instrument delivery device of FIG. 15;

FIG. 17 is a cross-sectional view of the instrument delivery device of FIG. 15 taken along the line 17-17, according to an aspect of the disclosure;

FIG. 18 is a cross-sectional view of the instrument delivery device of FIG. 15 taken along the line 17-17, according to another aspect of the disclosure;

FIG. 19 is a top view of an instrument delivery device, according to another aspect of the disclosure; and

FIG. 20 is a top view of a lock, and blunt cannula thereof, included in the instrument delivery device of FIG. 19.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device (e.g., the end of the device being manipulated by the user) would be the proximal end of the device.

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

The terms “first”, “second”, and the like are not intended to refer to any particular order or chronology, but refer to different conditions, properties, or elements.

As used herein, “at least one of” is synonymous with “one or more of”'. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

The present invention is directed to an instrument delivery device and introducer thereof that provides for advancement of an instrument into an indwelling catheter, with the introducer configured to extend the instrument out from the introducer by an increased length. The advancement of the instrument out from the introducer by a longer distance is achieved without increasing a length of the introducer, so as to keep the introducer at an acceptable size that allows for easy use thereof.

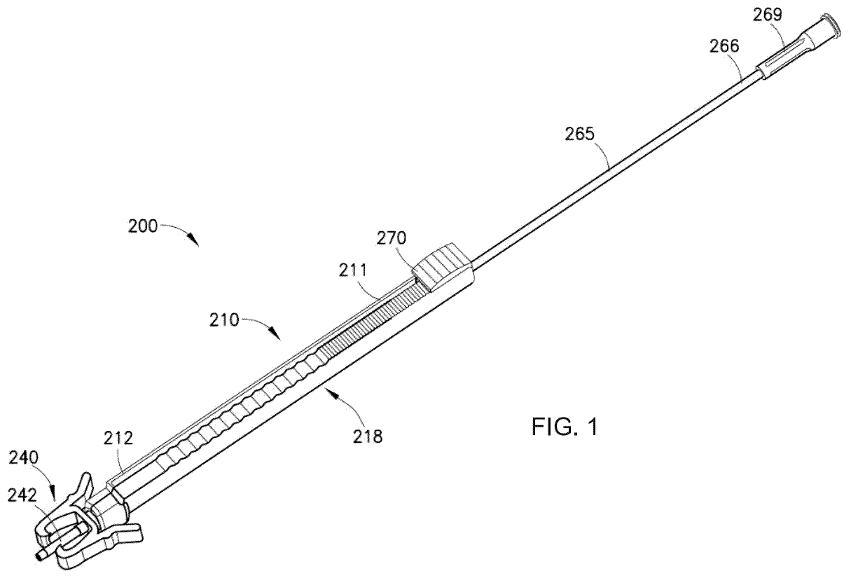

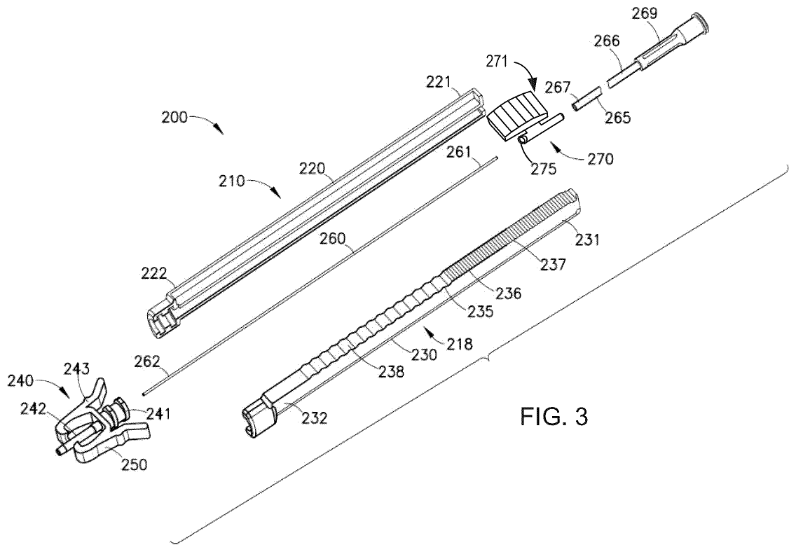

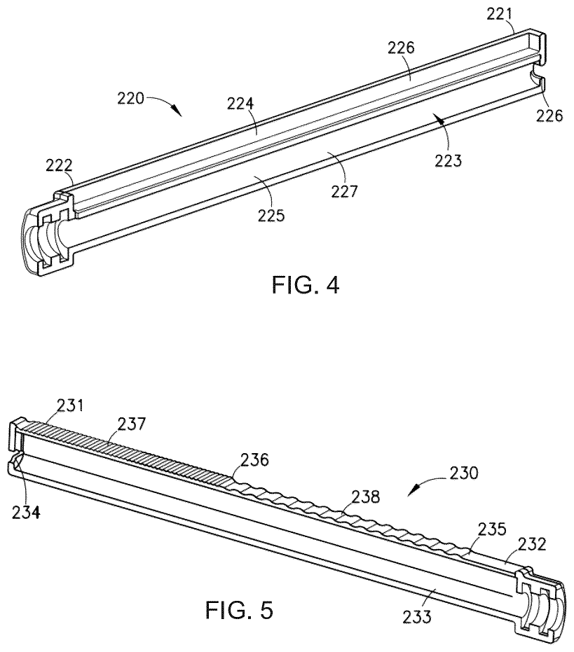

FIGS. 1-15 illustrate an instrument delivery device 200 and components thereof according to an embodiment of the disclosure. The instrument delivery device 200 can be any suitable shape, size, or configuration and can be coupled to a vascular access device including an indwelling catheter (not shown in FIGS. 1-15), for example, via a lock and/or adapter. As described in further detail herein, a user can transition the instrument delivery device 200 from a first configuration to a second configuration to advance an instrument through an existing, placed, and/or indwelling catheter (i.e., when the instrument delivery device 200 is coupled thereto) such that at least an end portion of the instrument is disposed in a distal position relative to the indwelling catheter. The instrument delivery device 200 can be manipulated by a user to place a distal surface of the instrument a predetermined and/or desired distance beyond a distal end of the indwelling catheter (e.g., 30-35 mm past the distal end, as a non-limiting example).

As shown in FIGS. 1-3, the instrument delivery device 200 includes an introducer 210, a lock 240, an instrument 260, an extension tube 265, and an actuator 270. The instrument 260 may be any of a number of devices suitable for insertion into the vasculature of the patient, including a catheter, guidewire, or probe, as examples. Thus, while for purposes of simplicity, the instrument 260 is referred to hereafter as a “tube 260” (i.e., a flexible polyimide tube/catheter), it is recognized that the instrument 260 could take the form of any of the aforementioned devices, according to aspects of the disclosure.

The introducer 210 can be any suitable shape, size, or configuration. For example, in some embodiments, the introducer 210 can be an elongate member having a substantially circular cross-sectional shape. In some embodiments, the shape of the introducer 210 and/or one or more features or surface finishes of at least an outer surface of the introducer 210 can be arranged to increase the ergonomics of the instrument delivery device 200, which in some instances, can allow a user to manipulate the instrument delivery device 200 with one hand (i.e., single-handed use).

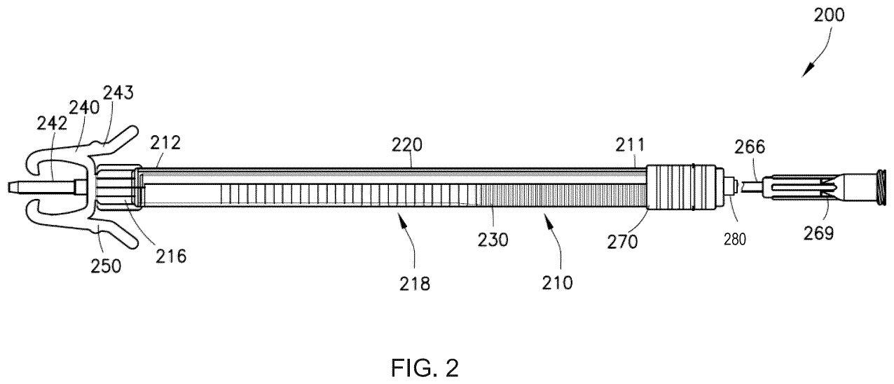

As shown in FIGS. 2-8, the introducer 210 of the instrument delivery device 200 includes an introducer housing 218 having a first housing member 220 and a second housing member 230 that are coupled to collectively form the housing 218. The first housing member 220 includes a proximal end portion 221, a distal end portion 222, and an inner surface 223. The inner surface 223 has a first portion 224 and a second portion 225, with second portion 225 being a flange in one embodiment. The proximal end portion 221 of the first housing member 220, and more specifically, a proximal wall of the first housing member 220 defines a notch 226 configured to selectively receive a portion of the extension tube 265, as described in further detail herein.

The second housing member 230 has a proximal end portion 231, a distal end portion 232, an inner surface 233, and a top surface 235. As described above with reference to the first housing member 220, the proximal end portion 231 of the second housing member 230, and more specifically, a proximal wall of the second housing member 230 defines a notch 234 configured to selectively receive a portion of the extension tube 265.

The top surface 235 of the second housing member 230 includes a set of ribs 236 distributed along a length of the second housing member 230, with each rib 236 extending along a width of the second housing member 230. The ribs 236 formed by the top surface 235 of the second housing member 230 can be any suitable shape, size, and/or configuration. In some embodiments, the set of ribs 236 may include a first portion 237 having a first size and shape, and a second portion 238 having a second size and shape, different from the first size and shape. Each rib in the second portion 238 has a size and shape that is greater than the size and shape of each rib of the first portion 237.

As described in further detail herein, a portion of the actuator 270 is configured to be advanced along the top surface 235 forming the set of ribs 236 as a user moves the actuator 270 relative to the introducer housing 218, which in turn, vibrates the actuator 270 (and the tube 260 coupled thereto). In some instances, this vibration can, for example, facilitate the advancing of the tube 260 through a portion or the instrument delivery device 200, a portion of the indwelling catheter, and/or a portion of the vasculature. In some instances, the greater size of the ribs of the second portion 238 can result in a larger amount of vibration as the actuator 270 is moved along the top surface 235 (as described above). In some instances, the greater size of the ribs of the second portion 238 can result in an increase in a force otherwise sufficient to move the portion of the actuator 270 along the top surface 235.

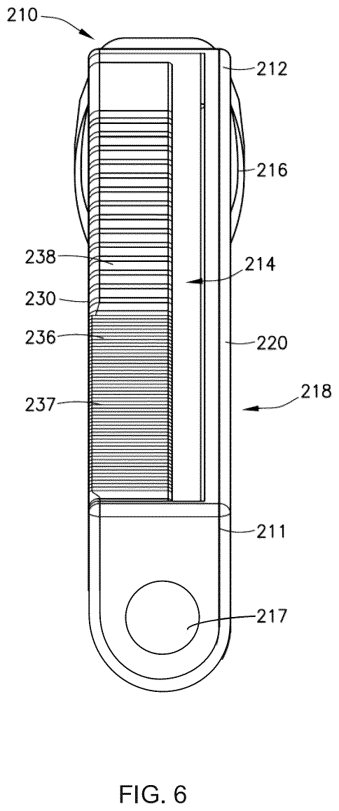

As shown in FIGS. 6-8, the first housing member 220 is configured to be coupled to the second housing member 230 to collectively form the introducer housing 218. In some embodiments, forming the introducer housing 218 from the first housing member 220 and the second housing member 230 can reduce undesirable variations in the shape and/or size of the inner surface 223 and 233 (e.g., due to draft angles and/or manufacturing tolerances) during manufacturing, which in some instances, can reduce a likelihood of kinks, bends, and/or deformations of the tube 260 during use of the instrument delivery device 200. In some embodiments, forming the introducer housing 218 from the first housing member 220 and the second housing member 230 can allow at least the inner surface 223 of the first housing member 220 to form a tortuous shape that would otherwise present challenges when manufacturing the introducer housing 218 from a single work piece.

The first housing member 220 and the second housing member 230 collectively form a proximal end portion 211 and a distal end portion 212 of the introducer housing 218 and collectively define an inner volume 213 of the introducer housing 218. As shown in FIG. 6, the proximal end portion 211 of the introducer housing 218 defines an opening 217. Specifically, the opening 217 is collectively formed and/or defined by the notch 226 of the first housing member 220 and the notch 234 of the second housing member 230. As described in further detail herein, the opening 217 is configured to receive a portion of the actuator 270 and/or extension tube 265, depending on the positioning of actuator 270.

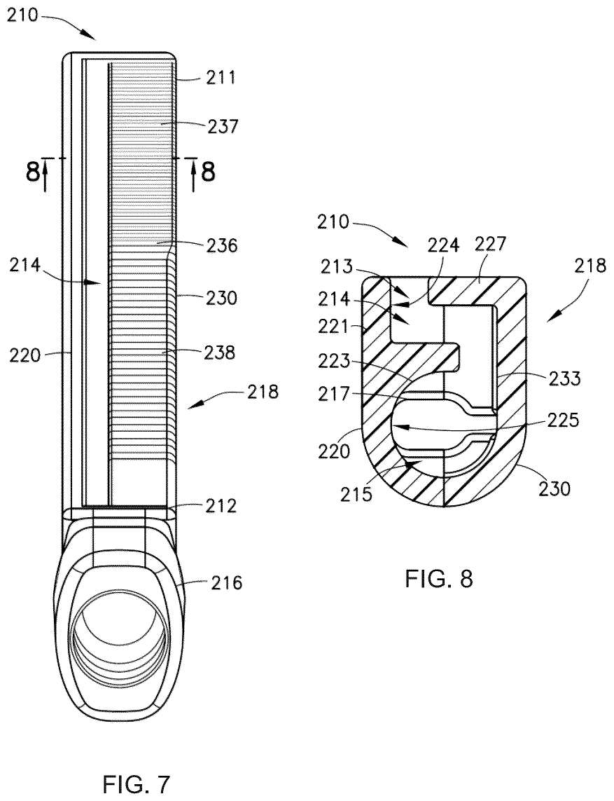

As shown in FIG. 7, the distal end portion 212 of the introducer housing 218 includes and/or otherwise forms a housing coupler 216. In other words, the distal end portion 222 of the first housing member 220 and the distal end portion 232 of the second housing member 230 collectively form the housing coupler 216 at the distal end portion 212 of the introducer housing 218. The housing coupler 216 can be any suitable shape, size, and/or configuration. For example, in the illustrated embodiment, the housing coupler 216 forms a set of inner-facing threads, which can form a threaded coupling with an associated threaded portion of the lock 240, as described in further detail herein. In other embodiments, the housing coupler 216 may include openings therein that receive snap-fit features formed on the lock 240.

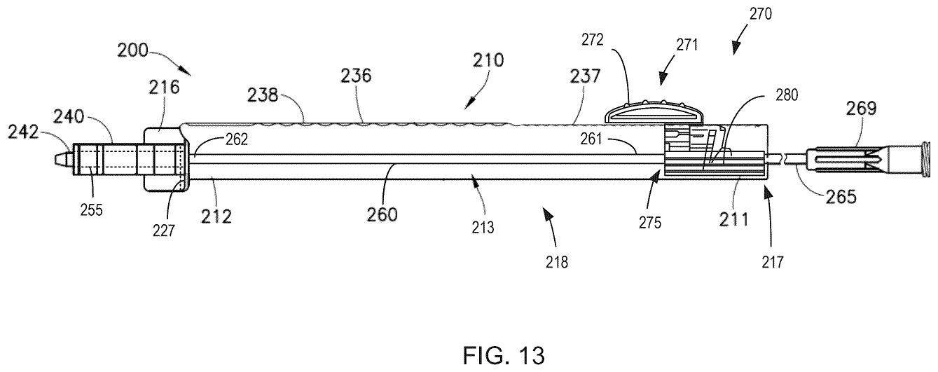

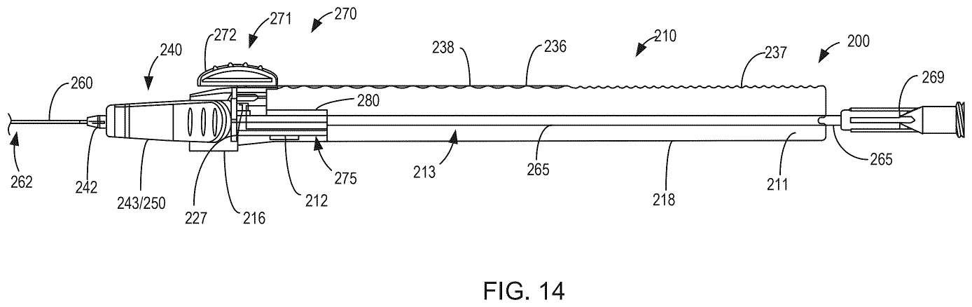

In some embodiments, the distal end portion 211 of the introducer housing 218 can include and/or can be configured to receive a seal 227 (see FIGS. 13 and 14) that can selectively seal and/or fluidically isolate the inner volume 213 of the introducer housing 218 (at least from an open portion of the housing coupler 216). In use, the seal 227 can be transitioned from a sealed or closed configuration to an open configuration to allow, for example, a portion of the tube 260 to pass therethrough. In some embodiments, the seal 227 can contact an outer surface of the tube 260 to define a seal therebetween that is operable to limit and/or substantially prevent a back flow of fluid between the outer surface of the cannula and the seal 227. The seal 227 may be placed within the housing coupler 216, such as by being seated in a groove formed in the housing coupler 216, and may be retained in place between upon the lock 240 being connected thereto (e.g., via a threaded engagement or snap-fit).

The inner surface 223 of the first housing member 220 and the inner surface 233 of the second housing member 230 collectively define the inner volume 213 of the introducer housing 218. As shown in FIG. 8, the arrangement of the inner surfaces 223 and 233 is such that the inner volume 213 has and/or defines a tortuous cross-sectional shape. For example, the inner volume 213 can have a substantially S-shaped or an at least partially S-shaped cross-sectional shape. More specifically, the inner surface 223 of the first housing member 220 includes and/or forms a ridge, tab, flange, protrusion, and/or wall configured to separate the first portion 224 of the inner surface 223 from the second portion 225 of the inner surface 223. Thus, the tortuous cross-sectional shape of the inner volume 213 forms and/or defines a first portion 214 of the inner volume 213 and a second portion 215 of the inner volume 213. In this manner, the first portion 214 of the inner volume 213 is spaced apart from the second portion 215 of the inner volume 213 without being fluidically isolated therefrom. In other words, the first portion 214 of the inner volume 213 defines an axis that is parallel to and offset from an axis defined by the second portion 215 of the inner volume 213.

As shown in FIG. 8, the first portion 214 of the inner volume 213 extends through a wall of the introducer housing 218. Similarly stated, the introducer housing 218 defines (e.g., the first housing member 220 and the second housing member 230 collectively define) a slot, channel, track, opening, and/or the like that is in fluid communication with the first portion 214 of the inner volume 213. Conversely, the second portion 215 of the inner volume 213 is entirely defined and/or enclosed (at least in the circumferential direction) by the introducer housing 218. The tortuous cross-sectional shape of the inner volume 213 is such that the second portion 215 cannot be viewed (e.g., is out of the line of sight) via the slot (in fluid communication with the first portion 214 of the inner volume 213), which in turn, can limit and/or substantially prevent contamination of the tube 260 disposed therein.

In this embodiment, the second portion 215 of the inner volume 213 is substantially aligned with, for example, a portion of the opening 217 and a portion of an opening defined by the housing coupler 216. Moreover, the second portion 215 of the inner volume 213 is configured to be substantially aligned with the lock 240 when the lock is coupled to the housing coupler 216 of the introducer housing 218. In other words, the axis defined by the second portion 215 of the inner volume 213 is substantially co-axial with an axis defined by a portion of the lock 240, as described in further detail herein. In this manner, the second portion 215 of the inner volume 213 can movably receive, for example, a portion of the actuator 270 and a portion of the tube 260. Thus, the actuator 270 can be moved relative to the introducer housing 218 to move the tube 260 between a first position, in which the tube 260 is entirely disposed within the second portion 215 of the inner volume 213, and a second position, in which at least a portion of the tube 260 extends outside of the second portion 215 of the inner volume 213 and distal to the introducer housing 218, as described in further detail herein.

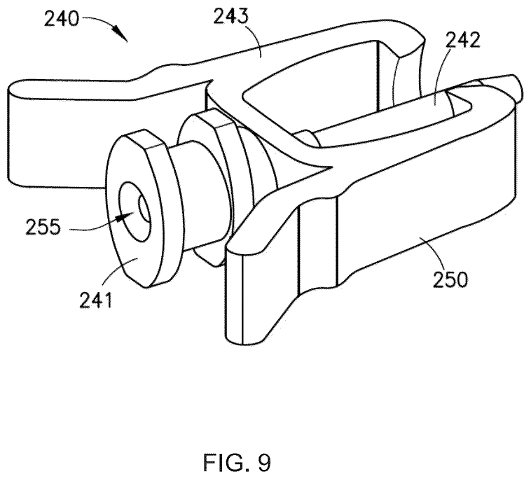

The lock 240 of the instrument delivery device 200 can be any suitable shape, size, and/or configuration. As described above, the lock 240 is configured to be physically and fluidically coupled to the introducer housing 218 and configured to couple the introducer 210 to the vascular access device including the indwelling catheter (i.e., to any suitable intermediate device or adapter of vascular access device coupled to the indwelling catheter). The lock 240 has a lock coupler 241, a blunt cannula 242, a first arm 243, and a second arm 250, as shown in FIG. 9. In addition, the lock 240 defines a lumen 255 extending through the lock coupler 241 and the blunt cannula 242. The lock coupler 241 is configured to couple the lock 240 to the housing coupler 216 of the introducer housing 218. Specifically, in this embodiment, the lock coupler 241 includes and/or forms one or more protrusions configured to selectively engage the threads defined and/or formed by the housing coupler 216 of the introducer housing 218, thereby forming a threaded coupling.

The blunt cannula 242 extends from the coupler 246 and is disposed between the first arm 243 and the second arm 250. The blunt cannula 242 can be any suitable shape, size, and/or configuration. In some embodiments, the blunt cannula 242 can have a length that is sufficient to extend through at least a portion of the PIVC or through an adapter and at least partially into or through the PIVC. Furthermore, the blunt cannula 242 can have an inner diameter (a diameter of a surface at least partially defining the lumen 255) that is similar to or slightly larger than an outer diameter of a portion of the tube 260. Thus, the lumen 255 of the lock 240 can receive a portion of the tube 260 when the instrument delivery device 200 is transitioned between the first configuration and the second configuration.

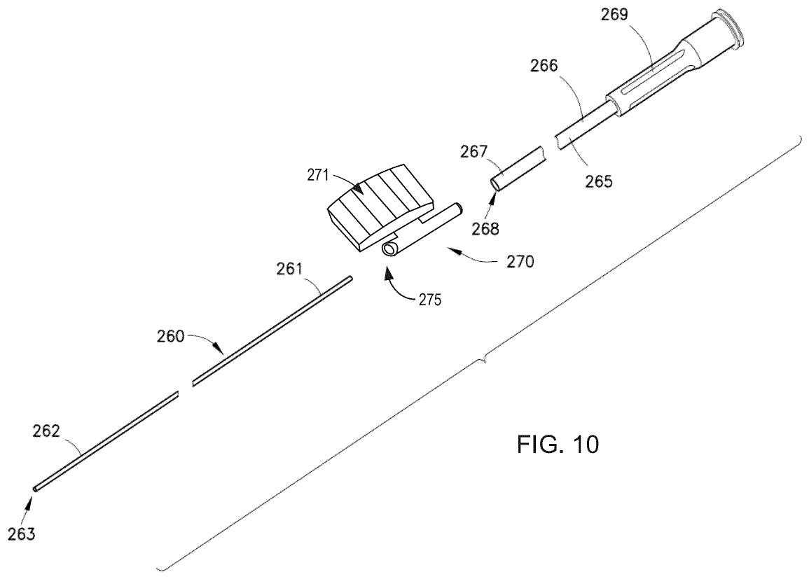

As described above, at least a portion of the tube 260 and at least a portion of the extension tube 265 is movably disposed within the second portion 215 of the inner volume 213 defined by the introducer housing 218. As shown in FIG. 10, the tube 260 has a proximal end portion 261 and a distal end portion 262 and defines a lumen 263. The proximal end portion 261 of the tube 260 is coupled to a second portion 275 of the actuator 270. In this manner, the actuator 270 can be moved relative to the introducer housing 218 to move the tube 260 between a first position, in which the tube 260 is disposed within the introducer housing 218 (e.g., the entire tube 260 is disposed within the introducer housing 218 or within the introducer housing 218 and the lock 240) and a second position, in which the distal end portion of the tube 260 is at least partially disposed in a position distal to the lock 240 and/or the PIVC (not shown) when the lock 240 is coupled to the PIVC, as described in further detail herein. The distal end portion 262 can be any suitable shape, size, and/or configuration and can define at least one opening in fluid communication with the lumen 263.

The extension tube 265 has a proximal end portion 266 and a distal end portion 267 and defines a lumen 268. As shown in FIG. 10, the proximal end portion 266 of the extension tube 265 is coupled to and/or otherwise includes a coupler 269. The coupler 269 is configured to physically and fluidically couple the extension tube 265 to any suitable device such as, for example, a fluid reservoir, fluid source, syringe, evacuated container holder (e.g., having a sheathed needle or configured to be coupled to a sheathed needle), pump, and/or the like. The distal end portion 267 of the extension tube 265 may be at least partially disposed within the second portion 215 of the inner volume 213 defined by the introducer housing 218 and is coupled to a second portion 275 of the actuator 270. In this manner, when the coupler 269 is coupled to a fluid reservoir, fluid source, syringe, evacuated container, pump, etc., the extension tube 265 establishes fluid communication between the reservoir, source, pump, etc. and the tube 260.

The actuator 270 of the instrument delivery device 200 is coupled to the tube 260 and can be moved along a length of the introducer housing 218 to transition the instrument delivery device 200 between its first configuration, in which the tube 260 is in the first position, and its second configuration, in which the tube 260 is in the second position. The actuator 270 can be any suitable shape, size, and/or configuration. For example, in some embodiments, the actuator 270 can have a size and shape that is associated with and/or based at least in part on a size and/or shape of the introducer housing 218.

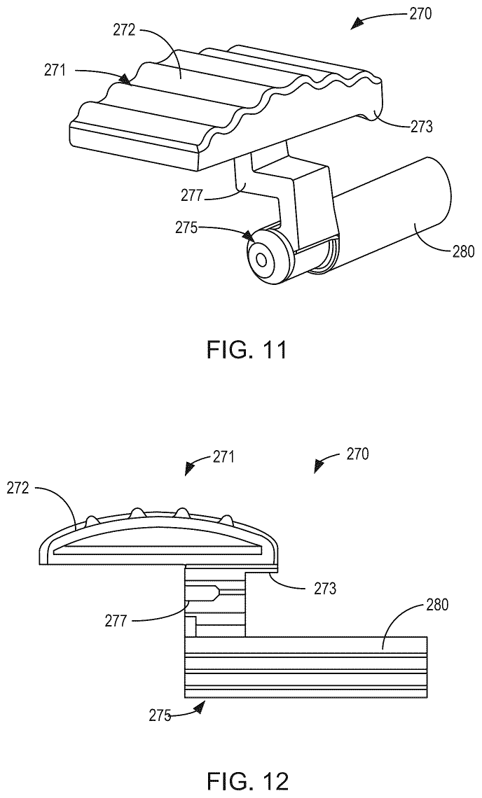

Referring now to FIGS. 11 and 12, the structure of actuator 270 is shown in greater detail. As shown in FIGS. 11 and 12, the actuator 270 includes a first portion 271, the second portion 275, and a wall 277 extending therebetween. The first portion 271 of the actuator 270 is at least partially disposed within the first portion 214 of the inner volume 213 (FIG. 8) defined by the introducer housing 218 and the second portion 275 of the actuator 270 is disposed within the second portion 215 of the inner volume 213 (FIG. 8), as described above. The first portion 271 of the actuator 270 includes an engagement member 272. The arrangement of the actuator 270 is such that the engagement member 272 is disposed outside of the introducer housing 218 while the rest of the first portion 271 is within the first portion 214 of the inner volume 213 defined by the introducer housing 218. As such, the engagement member 272 can be engaged and/or manipulated by a user (e.g., by a finger or thumb of the user) to move the actuator 270 relative to the introducer housing 218. In some embodiments, the engagement member 272 can include a set of ridges and/or any suitable surface finish that can, for example, increase the ergonomics of the actuator 270 and/or instrument delivery device 200.

The engagement member 272 includes a tab 273 disposed at or near a proximal end portion of the engagement member 272. The tab 273 can be any suitable tab, rail, ridge, bump, protrusion, knob, roller, slider, etc. that extends from a surface of the engagement member 272. The tab 273 is configured to selectively engage the top surface 235 of the second housing member 230 of the introducer housing 218. More specifically, the tab 273 is in contact with the ribs 236 formed by the second housing member 230 and moves along each successive rib as the actuator 270 is moved along a length of the introducer housing 218, with interaction of the tab 273 with the ribs 236 causing vibration of the actuator 270.

As shown in FIGS. 11 and 12, the actuator 270 is constructed such that the first portion 271 and the second portion 275 include sections thereof that extend out distally and proximally relative to each other, so as to increase an overall length and reach of the actuator 270. In particular, the engagement member 272 of the first portion 271 extends out distally relative to the second portion 275 and wall 277, such that the engagement member 272 is formed as a cantilevered engagement member, while the second portion 275 extends out proximally relative to the first portion 271 and wall 277, so as to form a proximally directed cylindrically-shaped extension 280 on actuator 270. With the actuator 270 configured as such, the range of motion of the tube 260 is increased—i.e., a length by which the tube 260 may be extended out from introducer 210 when in the second position is increased, as explained in greater detail below regarding FIGS. 13 and 14.

Referring now to FIGS. 13 and 14, changing of the instrument delivery device 200 between the first configuration and second configuration is shown. The instrument delivery device 200 can be in the first configuration prior to use and can be transitioned by a user (e.g., a doctor, physician, nurse, technician, phlebotomist, and/or the like) from the first configuration (FIG. 13) to the second configuration (FIG. 14) to dispose at least the distal end portion 262 of the tube 260 in a distal position relative to the introducer housing 218 (e.g., within an indwelling PIVC or distal to the indwelling PIV).

The instrument delivery device 200 is in the first configuration of FIG. 13 when the tube 260 is disposed in the first position within the introducer housing 218. In some embodiments, substantially the entire tube 260 is disposed within the housing 218 when the tube 260 is in the first position. In such embodiments, the introducer housing 218 can include a seal 227 or the like (as described above) that can substantially seal the distal end portion 212 of the housing 210 to isolate the tube 260 within the inner volume 213. In other embodiments, the tube 260 is disposed within the housing 210 and the lock 240 when tube 260 is in the first position.

As shown in FIG. 13, with the instrument delivery device 200 in the first configuration, the actuator 270 is disposed toward the proximal end portion 211 of the introducer 210. With the actuator 270 disposed toward the proximal end portion 211 of the introducer 210, the extension 280 of second portion 275 (that extends out proximally relative to the first portion 271 and wall 277) is positioned such that it protrudes out through the opening 217 formed in the proximal end portion 211 of the introducer housing 218, with the extension tube 265 thus coupled to the second portion 275 outside of the housing 218. The proximal end portion 261 of the tube 260 is coupled to the second portion 275 of the actuator 270 inside of the housing 218, and the tube 260 extends through the inner volume 213 of the introducer 210, with the distal end portion 262 of the tube 260 being received in the lumen of the housing coupler 216 and/or the lumen 255 of the lock 240.

To transition from the first configuration to the second configuration, the user engages the engagement member 272 of the first portion 271 of the actuator 270 to move the actuator 270 relative to the introducer housing 218, which in turn, moves the tube 260 from the first position (e.g., disposed within the introducer housing 218) toward the second position. In this manner, the tube 260 is moved through the inner volume 213 and the lumen 255 of the lock 240 and, as such, at least the distal end portion 262 of the tube 260 is disposed outside of and distal to the lock 240. In some instances, the tube 260 is considered to be in the second position when the actuator 270 is in a distal most position, with the distal end portion 262 of the tube 260 thus being placed in a desired position relative to a distal end portion of the indwelling catheter. In some instances, for example, a distal end 262 of the tube 260 can extend a predetermined distance beyond the distal end of the indwelling catheter (e.g., distal to the distal end of the indwelling catheter) when the tube 260 is in the second position, such that the distal end 262 of the tube 260 is positioned within the vein at a predetermined distance beyond the distal end of the indwelling catheter (e.g., a position within a vein that is substantially free from debris (e.g., fibrin/blood clots) otherwise surrounding the distal end portion of the indwelling catheter).

As shown in FIG. 14, with the instrument delivery device 200 in the second configuration, the actuator 270 is in a distal most position along the introducer housing 218 (i.e., at the distal end portion 212 of the housing 218). To provide for translation of the actuator 270 to this position, the set of ribs 236 provided on the introducer housing 218 extend to (or almost to) the distal end portion 212 of the housing 218. With the instrument delivery device 200 in the second configuration, the cantilevered portion of the engagement member 272 is positioned such that at least some of the cantilevered engagement member 272 extends or hangs over the housing coupler 216 and past a portion of the first and second arms 243, 250 of the lock 240. By advancing the actuator 270 to the distal most position along the introducer housing 218 in this manner, and with the actuator 270 having a cantilevered engagement member 272 as described above, the forward reach of the actuator 270 is increased—so that the amount/length by which the distal end portion 262 of the tube 260 may be disposed outside of and distal to the lock 240 (and out past a distal end of the indwelling catheter) may be maximized.

With the tube 260 in the second position (e.g., with the instrument delivery device 200 in the second configuration as shown in FIG. 14), the user can establish fluid communication between the vasculature of a patient and the tube 260. With the tube 260 in fluid communication with the vasculature, the instrument delivery device 200 can then aspirate a volume of blood from the vein based, at least in part, on disposing the distal end of the tube 260 at the predetermined and/or desired distance beyond the distal end of the indwelling catheter. In some instances, once a desired amount of blood has been collected and/or once a desired volume of a drug has been delivered to the patient, the user can move the actuator 270 in the proximal direction, such as moving the actuator 270 back to its proximal most position, so as to move the tube 260 back to the first position.

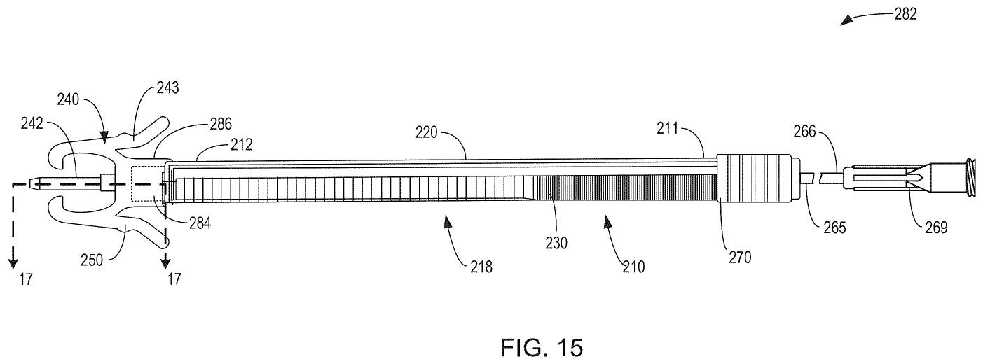

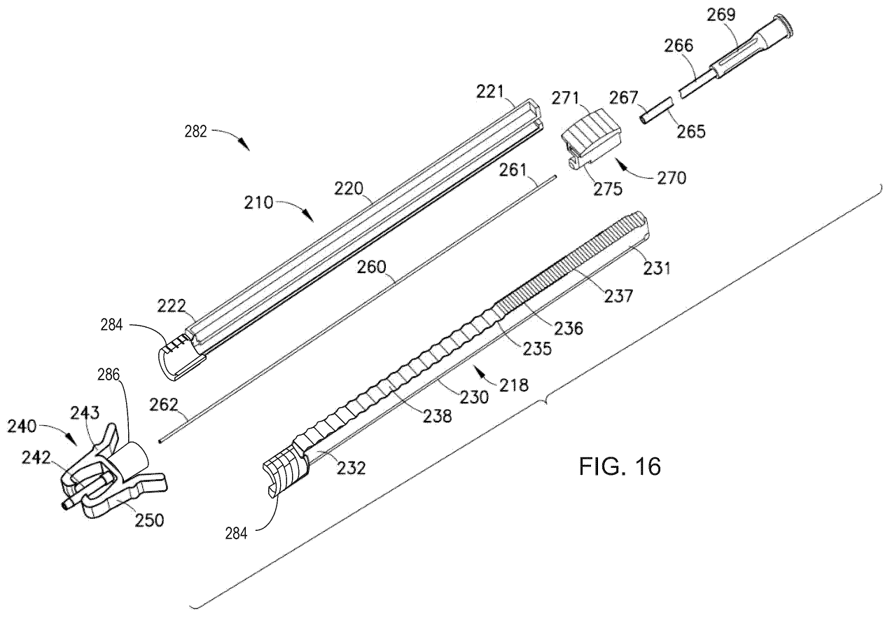

Referring now to FIGS. 15-17, an additional embodiment of an instrument delivery device 282 is illustrated, according to a further aspect of the disclosure. The instrument delivery device 282 is similar in construction to the instrument delivery device 200 of FIGS. 1-14, and thus identical components are numbered likewise to those components in FIGS. 1-14.

As explained in further detail below, the instrument delivery device 282 is configured so as to lengthen/maximize a reach of the instrument delivery device 282 (i.e., an amount by which an instrument, such as tube 260, may be advanced distally out therefrom), without changing the overall length of the introducer 210. However, instead of configuring actuator 270 with a cantilevered engagement member 272 and an extension member 280 (see FIGS. 11 and 12) to enable an increased range of movement thereof and a corresponding increased advancement length of the tube 260, the instrument delivery device 282 reconfigures the engagement between the introducer housing 218 and the lock 240 in order to increase the advancement length of the tube 260.

In the embodiments of FIGS. 15-17, the instrument delivery device 282 is reconfigured so as to create additional space at the distal end portion 212 of the introducer housing 218, so as to enable further advancement of actuator 270. Specifically, a housing coupler 284 of introducer housing 218 and a lock coupler 286 of lock 240 are configured such that the housing coupler 284 is at least partially positioned within the lock coupler 286 (rather than housing coupler 284 receiving/encircling the lock coupler 286, as in FIGS. 1-14), with an interior area/volume of the housing coupler 284 left open such that it may receive the second portion 275 of the actuator 270 therein.

As shown in FIGS. 15-17, the lock coupler 286 of lock 240 is configured to be positioned about the housing coupler 284 of introducer housing, with an inner diameter of the lock coupler 286 being larger than an outer diameter of the housing coupler 284. The lock coupler 286 may at least partially surround/encircle the housing coupler 284. According to one embodiment, the lock coupler 286 may be configured as a circular member that encircles the housing coupler 284, with the lock coupler 286 including a pair of radially inwardly directed protrusions 288 on opposing sides thereof that may snap-fit into engagement with openings 290 in the housing coupler 284—i.e., a U-shaped snap-fit.

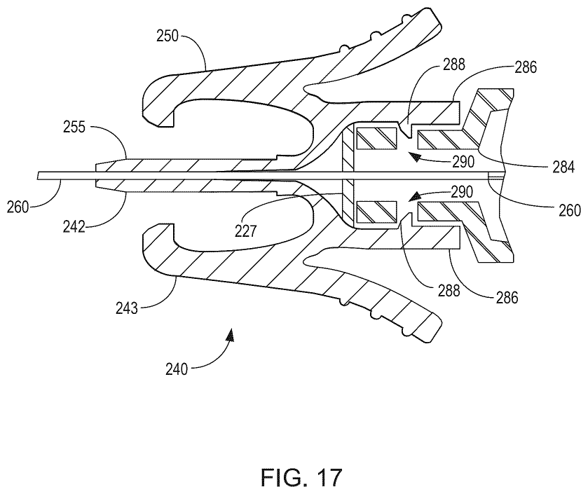

With the lock coupler 286 constructed as described above, the lock coupler 286 is able to accommodate the fluid flow preventing seal 227 therein (rather than having the seal 227 positioned within the proximal end of housing coupler 284, as shown in FIGS. 13 and 14). That is, as shown in FIG. 17, the seal 227 is fit within an inner volume (i.e., the lumen 255) defined by the lock coupler 286, with the seal 227 positioned toward a distal end of the lock coupler 286—where the lock coupler 286 transitions into the blunt cannula 242. The seal 227 may be seated in a groove formed in the lock coupler 286 and may be retained in place upon housing coupler 284 being mated with lock coupler 286 (e.g., via a snap-fit).

With the seal 227 positioned at the distal end of the lock coupler 286, the inner volume of the lock coupler 286 remains open to receive the housing coupler 284 therein, with the housing coupler 284 in-turn also remaining open (i.e., with no seal 227 positioned therein) to receive a portion of the actuator 270 (second portion 275) therein. With the instrument delivery device 282 in the second configuration and the actuator 270 advanced fully to the distal end portion 212 of the housing 210, at least some of the second portion 275 of the actuator 270 may enter into the interior of housing coupler 284. With the actuator 270 positioned as such, the forward reach of the actuator 270 is increased—so that the amount/length by which the distal end portion 262 of the tube 260 may be disposed outside of and distal to the lock 240 (and out past a distal end of the indwelling catheter) may be maximized.

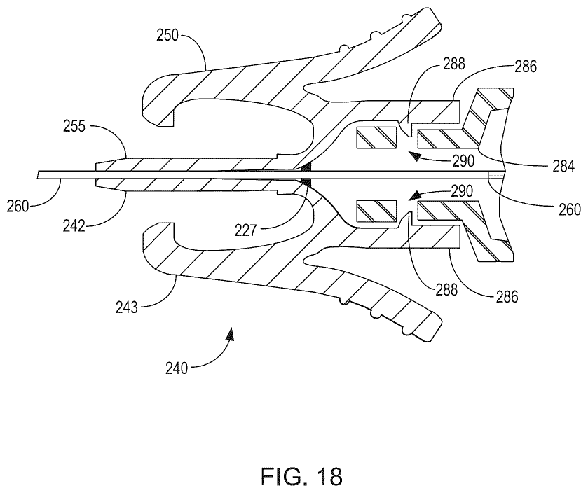

In another embodiment of instrument delivery device 282, and as shown in FIG. 18, rather than the seal 227 being positioned at the distal end of the lock coupler 286, the seal 227 may instead be positioned within a portion of the lumen 255 that is defined by the blunt cannula 242, such as the seal 227 being positioned within the lumen 255 toward the proximal end of the blunt cannula 242. With the seal 227 positioned within the lumen 255 of blunt cannula 242, the inner volume of the lock coupler 286 remains open to receive the housing coupler 284 therein, with the housing coupler 284 in-turn also remaining open (i.e., with no seal 227 positioned therein) to receive the second portion 275 of the actuator 270 therein when the instrument delivery device 282 is in the second configuration. Thus again, with the actuator 270 positioned as such, the forward reach of the actuator 270 is increased—so that the amount/length by which the distal end portion 262 of the tube 260 may be disposed outside of and distal to the lock 240 (and out past a distal end of the indwelling catheter) may be maximized.

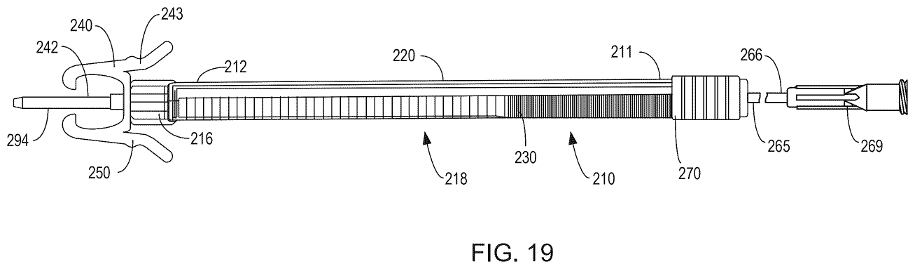

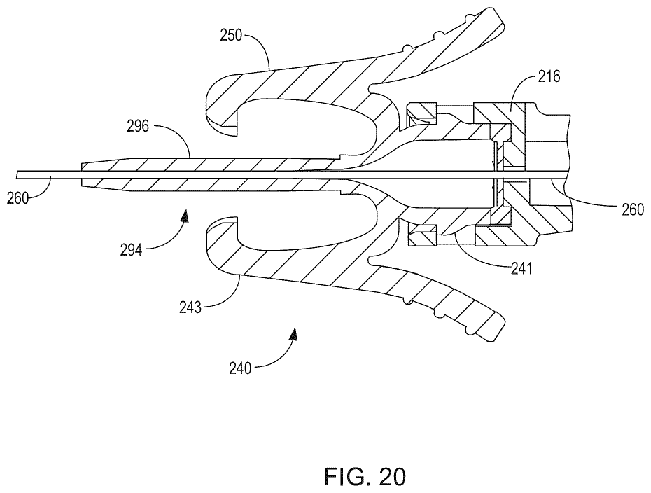

Referring now to FIGS. 19 and 20, yet another embodiment of an instrument delivery device 292 is illustrated, according to a further aspect of the disclosure. Again, the instrument delivery device 292 is similar in construction to the instrument delivery device 200, 282 of FIGS. 1-14 and FIGS. 15-18, and thus identical components are numbered likewise to those components in FIGS. 1-14.

As explained in further detail below, the instrument delivery device 292 is again configured so as to lengthen/maximize a reach of the instrument delivery device 292 (i.e., an amount by which an instrument, such as tube 260, may be advanced distally out therefrom), without changing the overall length of the introducer 210. However, instead of configuring actuator 270 with a cantilevered engagement member 272 and an extension member 280 and/or restructuring the lock coupler 286 and housing coupler 284 to enable an increased advancement length of the tube 260, the instrument delivery device 292 reconfigures the blunt cannula of lock 240 in order to increase the advancement length of the tube 260.

As shown in FIGS. 19 and 20, a length of a blunt cannula 294 of lock 240 is increased as compared to the blunt cannula 242 provided in the instrument delivery devices of FIGS. 1-14 and FIGS. 15-18. The blunt cannula 294 is lengthened such that it extends out substantially past the distal ends of the first arm 243 and second arm 250 of lock 240. In some embodiments, the blunt cannula 294 is configured such that 30% or more of a length of the blunt cannula 294 extends out distally past the distal ends of the first and second arms 243, 250 for engagement with an access connector (not shown) of a vascular access device. To enable such lengthening of the blunt cannula 294, a sidewall 296 of the blunt cannula 294 is constructed such that a thickness thereof is kept to 0.5 mm or more, such that the blunt cannula 294 retains sufficient strength to prevent breaking or bending thereof.

With the blunt cannula 294 having an increased length as described above, a length of the tube 260 included in instrument delivery device 292 can be increased. That is, as described previously, with the instrument delivery device 292 in the first configuration, the tube 260 is entirely positioned within the introducer housing 218 or within the introducer housing 218 and the lock 240 (i.e., the lumen 255 of lock 240). With the blunt cannula 294 having an increased length, a greater length of tube 260 can be accommodated within the introducer housing 218 and the lock 240, as the tube 260 can be positioned in the lengthened blunt cannula 294 when in the first position. Accordingly, a “starting position” of the tube 260 is made closer to the vascular access device (and the indwelling catheter thereof) and an extendible length of the tube 260 is increased by that same amount. When the actuator 270 is then actuated distally to transition the instrument delivery device 292 to the second configuration, the amount/length by which the distal end portion 262 of the tube 260 may be disposed outside of and distal to the lock 240 (and out past a distal end of the indwelling catheter) is thus also increased.

Beneficially, embodiments of the disclosure thus provide an instrument delivery device and introducer thereof that provides for advancement of an instrument into an indwelling catheter, with the introducer configured to extend the instrument out from the introducer by an increased length. The advancement of the instrument out from the introducer by a longer distance is achieved without increasing a length of the introducer, so as to keep the introducer at an acceptable size that allows for easy use thereof. The introducer allows for the instrument to be advanced into and through a catheter that is inserted further/deeper into the vascular (e.g., CVC or MIG catheter), and for the instrument to extend a further distance (e.g., 30-35 mm) past the distal tip of the indwelling catheter (e.g., to prevent thrombosis).

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. An instrument delivery device for advancing an instrument into an indwelling catheter of a vascular access device, the instrument delivery device comprising:
an introducer having an introducer housing comprising proximal end portion and a distal end portion, the distal end portion comprising a housing coupler, the introducer housing defining an inner volume configured to movably receive the instrument;
an actuator movably coupled to the introducer housing and configured to move relative to the introducer housing to move the instrument between a first position, in which the instrument is disposed within the introducer housing, and a second position, in which a distal end portion of the instrument is disposed beyond the distal end portion of the introducer housing, such that at least a portion of the instrument may be disposed within the indwelling catheter; and
a lock configured to secure the introducer housing to the vascular access device, the lock comprising:
a lock coupler configured to mate with the housing coupler of the introducer housing to secure the lock to the introducer housing, the lock coupler configured to be positioned at least partially about the housing coupler, such that the housing coupler is positioned within the lock coupler;
a blunt cannula extending out distally from the lock coupler and configured to engage an access connector of the vascular access device;
a pair of arms configured to engage the access connector; and
a fluid seal positioned within a lumen of the lock that extends through the lock coupler and the blunt cannula.

2. The instrument delivery device of claim 1, wherein the fluid seal is positioned within the lumen adjacent a distal end of the lock coupler, and distal to the housing coupler when mated with the lock coupler.

3. The instrument delivery device of claim 1, wherein the fluid seal is positioned within a portion of the lumen extending through the blunt cannula.

4. The instrument delivery device of claim 3, wherein the fluid seal is positioned at a proximal end of the lumen extending through the blunt cannula.

5. The instrument delivery device of claim 1, wherein the lock coupler comprises a U-shaped snap-fit that engages the housing coupler on opposing sides thereof.

6. The instrument delivery device of claim 1, wherein the actuator comprises a first portion and a second portion, the second portion positioned within the inner volume of the introducer housing and the first portion extending out through a slot formed in the introducer housing and onto a top surface of the introducer housing.

7. The instrument delivery device of claim 6, wherein the actuator is advanceable to the distal end of the introducer housing so that part of the second portion is positioned in the housing coupler, to place the instrument in the second position.

8. The instrument delivery device of claim 6, wherein the first portion of the actuator comprises an engagement member, and wherein the top surface of the introducer housing includes a plurality of ribs thereon that selectively engage an underside of the engagement member to provide for advancement of the actuator along the introducer housing.

9. The instrument delivery device of claim 8, wherein the plurality of ribs extend along the top surface of the introducer housing, to the distal end thereof.

10. An instrument delivery device for advancing an instrument into an indwelling catheter of a vascular access device, the instrument delivery device comprising:
an introducer comprising an introducer housing having a proximal end portion and a distal end portion, the distal end portion comprising a housing coupler, the introducer housing defining an inner volume configured to movably receive the instrument; and
an actuator movably coupled to the introducer housing and configured to move relative to the introducer housing to move the instrument between a first position, in which the instrument is disposed within the introducer housing, and a second position, in which a distal end portion of the instrument is disposed beyond the distal end portion of the introducer housing, such that at least a portion of the instrument may be disposed within the indwelling catheter;
wherein the actuator comprises:
a first portion comprising an engagement portion movable along a top surface of the introducer housing; and
a second portion positioned at least partially within the inner volume and joined to the first portion through a slot formed in the top surface of the introducer housing;
wherein the engagement member of the first portion is a cantilevered engagement member that extends distally out past the second portion.

11. The instrument delivery device of claim 10, wherein the second portion comprises a cylindrical extension that protrudes out proximally from the first portion.

12. The instrument delivery device of claim 11, wherein, with the instrument in the first position, the actuator is positioned such that the cylindrical extension extends out through an opening in the proximal end portion of the introducer housing.

13. The instrument delivery device of claim 12, further comprising an extension tube coupled to a proximal end of the cylindrical extension, the extension tube moving along with the actuator as the actuator is moved relative to the introducer housing.

14. The instrument delivery device of claim 10, wherein with the instrument in the second position, a portion of the cantilevered engagement member extends over the housing coupler.

15. The instrument delivery device of claim 14, wherein the top surface of the introducer includes a plurality of ribs thereon that selectively engage a tab on an underside of the engagement member to provide for advancement of the actuator along the introducer, and wherein the plurality of ribs extend along the top surface of the introducer, to the distal end thereof.

16. The instrument delivery device of claim 10, further comprising a lock configured to couple the introducer to the vascular access device, the lock comprising:
a lock coupler configured to mate with the housing coupler of the introducer to secure the lock to the introducer;
a blunt cannula extending out distally from the lock coupler and configured to engage an access connector of the vascular access device; and
a pair of arms positioned on opposing sides of the lock coupler and the blunt cannula and configured to engage the access connector.

17. The instrument delivery device of claim 16, wherein with the instrument in the second position, the cantilevered engagement member of the first portion extends over a portion of the pair of arms of the lock.

18. An instrument delivery device for advancing an instrument into an indwelling catheter of a vascular access device, the instrument delivery device comprising:
an introducer comprising an introducer housing having a proximal end portion and a distal end portion, the distal end portion comprising a housing coupler, the introducer housing defining an inner volume configured to movably receive the instrument;
a lock configured to secure the introducer to the vascular access device, the lock comprising:
a lock coupler configured to mate with the housing coupler of the introducer housing to secure the lock to the introducer housing;
a blunt cannula extending out distally from the lock coupler and configured to engage an access connector of the vascular access device; and
a pair of arms positioned on opposing sides of the lock coupler and the blunt cannula, each of the pair of arms comprising a first end configured to engage the access connector and a second end actuatable by a user to cause the first end to selectively engage and disengage the access connector;
wherein the blunt cannula comprises an extended cannula configured such that 30% or more of the blunt cannula extends out distally past the first end of the pair of arms for engagement with the access connector; and
an actuator movably coupled to the introducer housing and configured to move relative to the introducer housing to move the instrument between a first position, in which the instrument is disposed within the introducer housing and the lock, and a second position, in which a distal end portion of the instrument is disposed beyond the distal end portion of the introducer housing, such that at least a portion of the instrument may be disposed within the indwelling catheter.

19. The instrument delivery device of claim 18, wherein the blunt cannula has a sidewall defining a lumen therethrough, and wherein a thickness of the sidewall is 0.5 mm or more.

20. The instrument delivery device of claim 18, wherein with the instrument in the first position, a distal end of the instrument is positioned within a lumen of the blunt cannula.
